(19) United States
(12) Patent Application Publication
(10) Pub. No.: US 2020/0230306 A1
(43) Pub. Date: Jul. 23, 2020

(54) VENTRICULAR ASSIST BLOOD PUMP

(71) Applicant: SUN MEDICAL TECHNOLOGY RESEARCH CORPORATION, Nagano (JP)

(72) Inventors: Shinji KOBAYASHI, Nagano (JP); Naofumi MIYAJIMA, Nagano (JP); Eisuke KUSANO, Nagano (JP)

(21) Appl. No.: 16/650,872

(22) PCT Filed: Sep. 29, 2017
(86) PCT No.: PCT/JP2017/035703

§ 371 Date: Mar. 26, 2020

(51) Int. Cl. A61M 1/10; A61M 1/12

(52) U.S. Cl. CPC A61M 1/101; A61M 1/1086; A61M 2205/3334; A61M 1/122; A61M 1/1005

(57) ABSTRACT

A ventricular assist blood pump includes: a rotary part, a base part, and a housing. A value obtained by dividing an outer profile volume of the rotary part by a rotary volume of the rotary part is from 0.1 to 0.4. Assuming a volume which is a portion of a volume of a pump chamber and is disposed on a side where the rotary part exists obtained by partitioning by a predetermined plane P as a pump chamber main volume, a value obtained by dividing a rotary volume of the rotary part by the pump chamber main volume is from 0.4 to 0.7. A numerical value obtained by dividing the outer profile volume of the rotary part by the pump chamber main volume is from 0.05 to 0.2.

### RELATED APPLICATIONS

The present application is a National Phase of International Application Number PCT/JP2017/035703, filed Sep. 29, 2017.

### TECHNICAL FIELD

The present invention relates to a ventricular assist blood pump.

### BACKGROUND ART

Conventionally, there have been known a ventricular assist system and a ventricular assist blood pump which assist functions of a heart (for example, see patent literature 1).

FIG. 5 is an exploded perspective view for describing a conventional ventricular assist blood pump 900. As shown in FIG. 5, the conventional ventricular assist blood pump 900 includes: a rotary part 910 which has an impeller 912, a base part 920 which rotatably supports the rotary part 910; and a housing 930 which forms a pump chamber for housing the rotary part 910 in combination with the base part 920.

The conventional ventricular assist blood pump 900 can, as a main constitutional element of the ventricular assist system, assist functions of a heart of a user who is a heart disease patient during a period until a heart transplant is performed.

### CITATION LIST

### Patent Literature

PTL1: JP 2009-523488 A

### SUMMARY OF INVENTION

### Technical Problem

The treatment of a heart disease is extremely difficult and hence, at present, in many cases, there is no fundamental treatment method other than a heart transplant.

However, it is a rare case that conditions necessary for performing a heart transplant are satisfied readily. Accordingly, a current situation is that a heart disease patient waiting for a heart transplant (transplant waiting patient) has to wait for a long period until the patient can receive a heart transplant. Accordingly, a period until the patient can receive a heart transplant becomes extremely long and, eventually, there may be a case where a patient cannot receive a heart transplant operation.

In view of the above-mentioned circumstances, there has been proposed an idea where a patient does not receive a heart transplant operation, and uses a ventricular assist blood pump throughout his life.

In this case, there arises a tendency that a period during which a user who uses a ventricular assist blood pump (hereinafter simply referred to as “patient”) uses the ventricular assist blood pump becomes longer than an estimated period. As a result, the importance of suppressing a degree of worsening of a health condition of a user when a ventricular assist blood pump is used for a long period is further increased.

The present invention has been made in view of the above-mentioned circumstance, and it is an object of the present invention to provide a ventricular assist blood pump which can suppress a degree of worsening of a health condition of a user when the ventricular assist blood pump is used for a long period compared to a conventional ventricular assist blood pump.

### Solution to Problem

[1] According to an aspect of the present invention, there is provided a ventricular assist blood pump which includes:

a rotary part which has an impeller; a base part which rotatably supports the rotary part; and a housing which forms a pump chamber which houses the rotary part in combination with the base part, wherein a numerical value obtained by dividing an outer profile volume of the rotary part by a rotary volume of the rotary part is set to a value which falls within a range of from 0.1 to 0.4.

According to the ventricular assist blood pump of the present invention, the numerical value obtained by dividing the outer profile volume of the rotary part by the rotary volume of the rotary part is 0.4 or less and hence, an efficiency that the ventricular assist blood pump discharges blood can be increased by making the outer profile volume of the rotary part sufficiently small and hence, a rotational speed of the rotary part for obtaining a required flow rate can be reduced. As a result, the ventricular assist blood pump of the present invention becomes a ventricular assist blood pump which can suppress a degree of worsening of a health condition of a user when the ventricular assist blood pump is used for a long period compared to a conventional ventricular assist blood pump.

A relationship between “a rotational speed of the rotary part for obtaining a required flow rate can be reduced” and “can suppress a degree of worsening of a health condition of a user when the ventricular assist blood pump is used for a long period” is described hereinafter.

When a rotational speed of the rotary part for obtaining a required flow rate can be reduced, a rotational speed of the rotary part can be lowered and hence, a load which the rotary part applies to blood in an in-use state can be made small. In this case, a drawback brought about due to shearing or stirring of blood caused by blood which flows at a high speed such as, for example, complications such as hemolysis or bleeding in a digestive tract minimally occurs. Accordingly, with the use of the ventricular assist blood pump having the above-mentioned technical features, it is possible to suppress a degree of worsening of a health condition of a user when the ventricular assist blood pump is used for a long period.

In the ventricular assist blood pump of the present invention, it is preferable that the numerical value obtained by dividing the outer profile volume of the rotary part by the rotary volume of the rotary part be set to 0.3 or less. With such a configuration, an efficiency that the ventricular assist blood pump discharges blood can be further increased by making the outer profile volume of the rotary part further smaller and hence, a rotational speed of the rotary part for obtaining a required flow rate can be further reduced.

In making the outer profile volume of the rotary part sufficiently small based on the design of the rotary part of a conventional ventricular assist blood pump, it is preferable to prevent a ratio that a volume of blades of an impeller occupies in the whole volume of the rotary part from becoming smaller than a corresponding ratio of a conventional rotary part (by decreasing with priority a volume of a rotational symmetrical portion of a rotary part, for example, a volume of an impeller proximal portion).

Further, according to the ventricular assist blood pump of the present invention, a numerical value obtained by dividing an outer profile volume of the rotary part by a rotary volume of the rotary part is set to 0.1 or more and hence, it is possible to sufficiently ensure a strength of the impeller.

In the ventricular assist blood pump according to the present invention, it is more preferable that the numerical value obtained by dividing the outer profile volume of the rotary part by the rotary volume of the rotary part be set to 0.2 or more. With such a configuration, it is possible to further increase a strength of the impeller with a margin.

[2] According to another aspect of the present invention, there is provided a ventricular assist blood pump which includes: a rotary part which has an impeller; a base part which rotatably supports the rotary part; and a housing which forms a pump chamber which houses the rotary part in combination with the base part, wherein a suction port which is disposed on an axis of rotation of the impeller and a discharge port which is disposed on an outer peripheral side of the impeller are formed in the housing, and assuming a plane which is perpendicular to the axis of rotation of the impeller and includes a distal end of the rotary part on a suction port side during an operation time of the ventricular assist blood pump as a predetermined plane and assuming a volume which is a portion of a volume of the pump chamber disposed on a side where the rotary part exists and is obtained by partitioning by the predetermined plane as a pump chamber main volume, a numerical value obtained by dividing a rotary volume of the rotary part by the pump chamber main volume is set to a value which falls within a range of from 0.4 to 0.7.

According to the ventricular assist blood pump of the present invention, the numerical value obtained by dividing the rotary volume of the rotary part by the pump chamber main volume is 0.4 or more and hence, an efficiency that the ventricular assist blood pump discharges blood can be increased by making the rotary volume of the rotary part sufficiently large compared to the pump chamber main volume whereby a rotational speed of the rotary part for obtaining a required flow rate can be reduced. As a result, the ventricular assist blood pump of the present invention becomes a ventricular assist blood pump which can suppress a degree of worsening of a health condition of a user when the ventricular assist blood pump is used for a long period compared to a conventional ventricular assist blood pump.

In the ventricular assist blood pump of the present invention, it is preferable that the numerical value obtained by dividing the rotary volume of the rotary part by the pump chamber main volume be 0.5 or more. With such a configuration, an efficiency that the ventricular assist blood pump discharges blood can be increased by making the rotary volume of the rotary part sufficiently larger compared to the pump chamber main volume and hence, a rotational speed of the rotary part for obtaining a required flow rate can be further reduced.

Further, in the ventricular assist blood pump according to the present invention, it is more preferable that the numerical value obtained by dividing the rotary volume of the rotary part by the pump chamber main volume be 0.7 or less. Accordingly, a ratio that the rotary part occupies in the main portion of the pump chamber when the ventricular assist system is used can be set to a proper value and hence, both a volume of a portion in the pump chamber which is not occupied by the rotary part and a volume of a portion in the pump chamber where the rotary part does not pass can respectively have a margin. As a result, a stress applied to sucked blood can be sufficiently reduced, and it is possible to prevent the flow of blood from the suction port to the discharge port from being obstructed by the rotary part by itself.

In the ventricular assist blood pump according to the present invention, it is more preferable that the numerical value obtained by dividing the rotary volume of the rotary part by the pump chamber main volume be 0.65 or less. With such a configuration, a ratio that the rotary part occupies in the main portion of the pump chamber when the ventricular assist system is used can be set to a more proper value and hence, both a volume of a portion in the pump chamber which is not occupied by the rotary part and a volume of a portion in the pump chamber where the rotary part does not pass can respectively have a more margin. As a result, a stress applied to sucked blood can be further reduced, and it is possible to more effectively prevent the flow of blood from the suction port to the discharge port from being obstructed by the rotary part by itself.

[3] According to another aspect of the present invention, there is provided a ventricular assist blood pump which includes: a rotary part which has an impeller; a base part which rotatably supports the rotary part; and a housing which forms a pump chamber which houses the rotary part in combination with the base part, wherein a suction port which is disposed on an axis of rotation of the impeller and a discharge port which is disposed on an outer peripheral side of the impeller are formed in the housing, and assuming a plane which is perpendicular to the axis of rotation of the impeller and includes a distal end of the rotary part on a suction port side during an operation time of the ventricular assist blood pump as a predetermined plane and assuming a volume which is a portion of a volume of the pump chamber and is disposed on a side where the rotary part exists obtained by partitioning by the predetermined plane as a pump chamber main volume, a numerical value obtained by dividing an outer profile volume of the rotary part by the pump chamber main volume is set to a value which falls within a range of from 0.05 to 0.2.

According to the ventricular assist blood pump of the present invention, the numerical value obtained by dividing the outer profile volume of the rotary part by the pump chamber main volume is set to 0.2 or less and hence, a pressure loss can be sufficiently reduced whereby it is possible to sufficiently increase a change in a flow rate against a change in a head. As a result, the ventricular assist blood pump of the present invention becomes a ventricular assist blood pump which can suppress a degree of worsening of a health condition of a user when the ventricular assist blood pump is used for a long period compared to a conventional ventricular assist blood pump.

A relationship between “a pressure loss can be sufficiently reduced whereby it is possible to sufficiently increase a change in a flow rate against a change in a head” and “can suppress a degree of worsening of a health condition of a user when the ventricular assist blood pump is used for a long period” is described hereinafter.

In the case where “a pressure loss can be sufficiently reduced whereby it is possible to sufficiently increase a change in a flow rate against a change in a head”, when the ventricular assist blood pump is driven under predetermined conditions (by fixing inputting conditions), pulsatility of a patient own heart is easily reflected on a change in a flow of blood. Accordingly, a defect caused by the reduction of pulsatility of flow of blood such as, for example, complications such as aortic regurgitation minimally occurs. As a result, the ventricular assist blood pump having the above-mentioned technical features can “suppress a degree of worsening of a health condition of a user when the ventricular assist blood pump is used for a long period”.

In the ventricular assist blood pump according to the present invention, it is further preferable that the numerical value obtained by dividing the outer profile volume of the rotary part by the pump chamber main volume be set to 0.17 or less. With such a configuration, a pressure loss can be sufficiently reduced and hence, it is possible to sufficiently increase a change in a flow rate against a change in a head.

Further, in the ventricular assist blood pump according to the present invention, the numerical value obtained by dividing the outer profile volume of the rotary part by the pump chamber main volume is set to 0.05 or more and hence, it is possible to sufficiently ensure a flow rate of blood (to sufficiently ensure a force which the rotary part moves blood).

In the ventricular assist blood pump of the present invention, it is more preferable that the numerical value obtained by dividing the outer profile volume of the rotary part by the pump chamber main volume be set to 0.1 or more. With such a configuration, it is possible to allow a flow rate to have a more margin (to allow the rotary part to have a more margin in a force for moving blood).

[4] In the ventricular assist blood pump according to the present invention, it is preferable that a minimum distance between the impeller and the housing during the operation of the ventricular assist blood pump be set to a value which falls within a range of from 0.2 mm to 1.0 mm.

With such a configuration, since the minimum distance between the impeller and the housing during the operation of the ventricular assist blood pump is 0.2 mm or more, a pressure loss can be sufficiently lowered. Further, since the minimum distance is 1.0 mm or less, it is possible to sufficiently ensure a force generated by the impeller for discharging blood.

[5] In the ventricular assist blood pump according to the present invention, it is preferable that a minimum distance between the impeller and the base part during the operation of the ventricular assist blood pump be set to a value which falls within a range of from 0.1 mm to 0.9 mm.

With such a configuration, since the minimum distance between the impeller and the base part during the operation of the ventricular assist blood pump is 0.1 mm or more, a pressure loss can be sufficiently lowered. Further, since the minimum distance is 0.9 mm or less, it is possible to more sufficiently ensure a force generated by the impeller for discharging blood.

[6] In the ventricular assist blood pump according to the present invention, it is preferable that, in a measurement of a pressure loss where a liquid having viscosity and density equivalent to viscosity and density of blood is used as a working liquid and a flow rate is set to 6 L/min in a state where the ventricular assist blood pump is stopped, the pressure loss be 20 mmHg or less.

With such a configuration, the pressure loss can be sufficiently lowered.

[7] In the ventricular assist blood pump according to the present invention, it is preferable that the ventricular assist blood pump be a centrifugal ventricular assist blood pump.

With such a configuration, the ventricular assist blood pump can more efficiently suck and discharge blood compared to an axial ventricular assist blood pump.

### BRIEF DESCRIPTION OF DRAWINGS

FIG. 1 is a view showing a mode where a ventricular assist blood pump 110 according to the embodiment is actually used;

FIGS. 2A and 2B are views for describing the ventricular assist blood pump 110 according to the embodiment;

FIG. 3 is a view for describing constitutional elements in the vicinity of a pump chamber of the ventricular assist blood pump 110 according to the embodiment; and

FIGS. 4A, 4B and 4C are views for describing an impeller 12 of a rotary part 10 according to the embodiment; and

FIG. 5 is an exploded perspective view for describing a conventional ventricular assist blood pump 900.

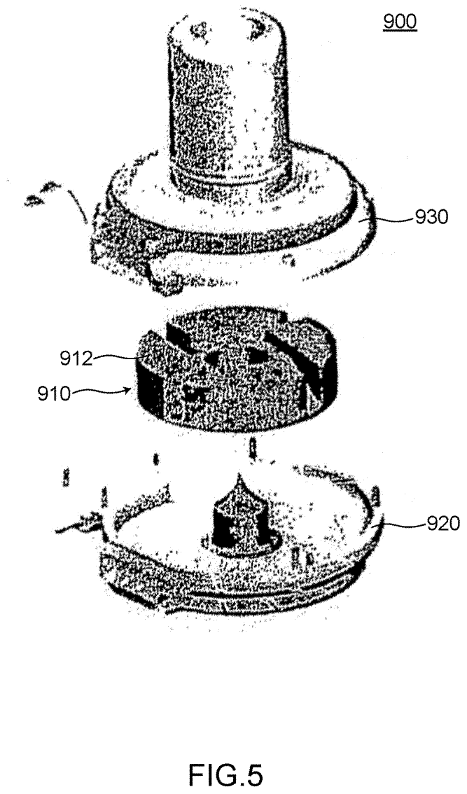

### DESCRIPTION OF EMBODIMENTS

Hereinafter, a ventricular assist blood pump according to the present invention is described based on an embodiment shown in the drawings. The embodiment described hereinafter is not limited to claims. Further, it is not always the case where the entirety of various constitutional elements and the combination of these constitutional elements described in the embodiment are indispensable for the present invention to overcome the drawbacks.

### Embodiment

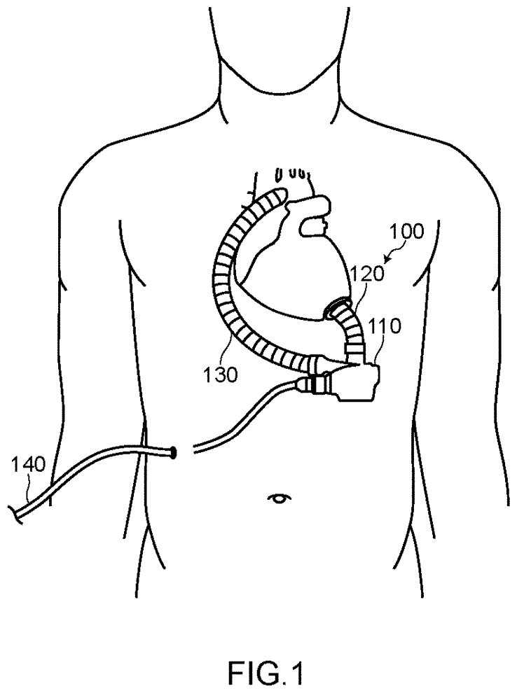

FIG. 1 is a view for describing a mode where a ventricular assist blood pump 110 according to the embodiment is actually used.

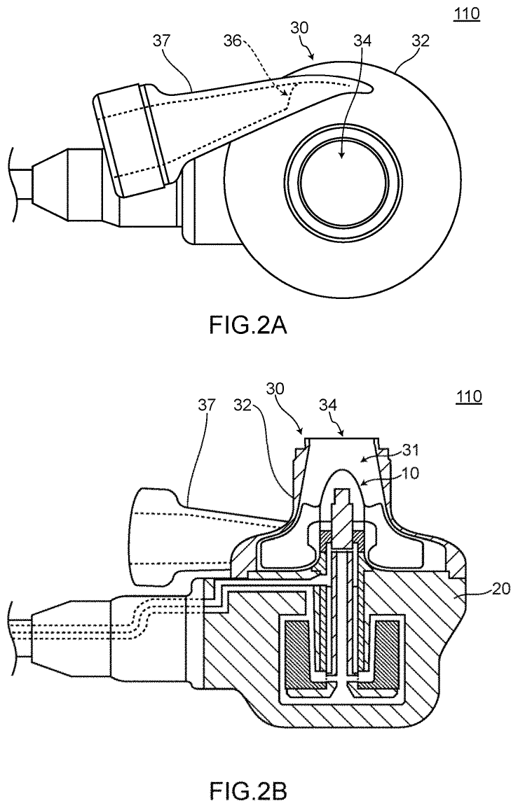

FIGS. 2A and 2B are views for describing the ventricular assist blood pump 110 according to the embodiment. FIG. 2A is a top plan view of the ventricular assist blood pump 110, and a FIG. 2B is a cross-sectional view of the ventricular assist blood pump 110.

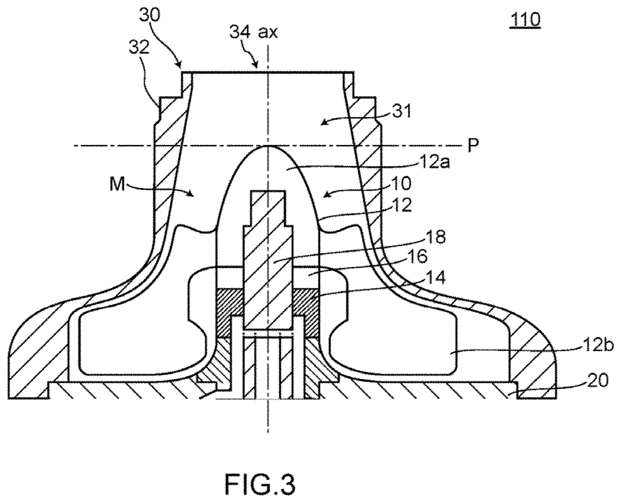

FIG. 3 is a view for describing constitutional elements of the ventricular assist blood pump 110 according to the embodiment in the vicinity of a pump chamber 31.

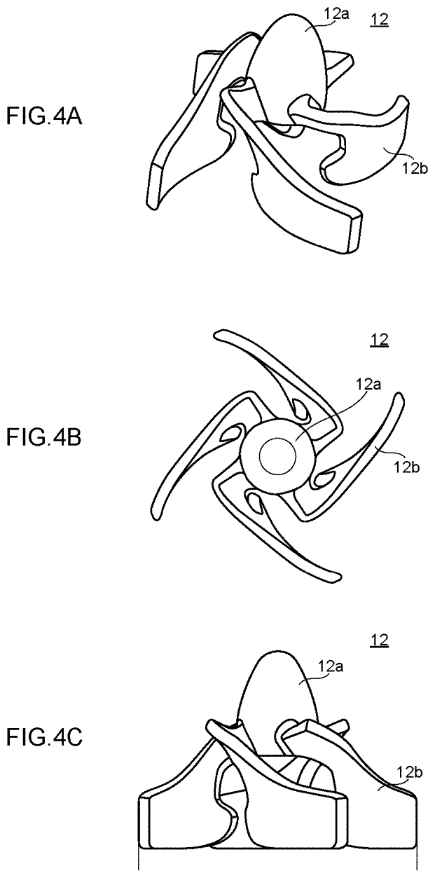

FIGS. 4A, 4B and 4C are views for describing an impeller 12 of a rotary part 10 according to the embodiment. FIG. 4A is a perspective view of the impeller 12, FIG. 4B is a top plan view of the impeller 12, and FIG. 4C is a front view of the impeller 12.

As shown in FIG. 1, the ventricular assist blood pump 110 according to the embodiment forms a part of the ventricular assist system 100. The ventricular assist system 100 includes, besides the ventricular assist blood pump 110, artificial blood vessels 120, 130, a cable 140 and a control part (not shown in the drawings). The control part is connected to the ventricular assist blood pump 110 via the cable 140, and controls an operation of the ventricular assist blood pump 110.

As shown in FIGS. 2A, 2B and FIG. 3, the ventricular assist blood pump 110 is a centrifugal ventricular assist blood pump which includes: a rotary part 10 having an impeller 12; a base part 20 which rotatably supports the rotary part 10; and a housing 30 which forms a pump chamber 31 for housing the rotary part 10 in combination with the base part 20. The ventricular assist blood pump 110 is an embedded-type ventricular assist blood pump which is used in a state where the ventricular assist blood pump 110 is embedded in a human body in an actual in-use state.

In the base part 20 of the ventricular assist blood pump 110, a driving part which rotatably drives the rotary part 10, fluid paths for a cooling seal liquid (also referred to as a purge liquid, for example, water or a saline) having functions such as lubrication, cooling, maintaining of a sealing property of the inside of the ventricular assist blood pump 110, and the like are disposed. These constitutional elements do not directly relate to the present invention, and hence, the description of these constitutional elements and the indication of symbols of these constitutional elements in the drawings are omitted.

In this specification, “rotary part” means a rotary part which rotates in the pump chamber in an in-use state of the ventricular assist blood pump 110. Further, in this specification, with respect to parts which are disposed astride the inside and the outside of the pump chamber and rotates in an in-use state of the ventricular assist blood pump 110 (for example, a rotary shaft), only a portion of the part which is disposed in the pump chamber is treated as the rotary part.

In this specification, “base part” is a part which restricts the position of the rotary part by any suitable means, and is fixed without being rotated in an in-use state of the ventricular assist blood pump 110. It is sufficient that (an outer frame) of the base part is not rotated as a whole in an in-use state of the ventricular assist blood pump 110. The base part may include a constitutional element which is operable in an in-use time of the ventricular assist blood pump 110 (for example, a mechanism or device which rotates the rotary part) in a portion of (in the inside of) the base part.

In this specification, with respect to the base part, “rotatably supports the rotary part” is not limited to a case where the base part and the rotary part are mechanically connected to each other. Even in a case where the base part and the rotary part are not brought into contact with each other in an in-use state of the ventricular assist blood pump 110 (in a case where the ventricular assist blood pump is a magnetic floating type ventricular assist blood pump), “rotatably supports the rotary part” also includes such a case provided that the base part has a mechanism which restricts the position of the rotary part.

Besides the impeller 12, the rotary part 10 further includes a seal ring 14 which forms a mechanical seal together with a member mounted on the base part 20, a cushion ring 16 which is interposed between the seal ring 14 and the impeller 12, and a rotary shaft 18 which extends from the driving part of the base part 20. In an in-use time of the ventricular assist blood pump 110, a rotational speed of the rotary part 10 can be set to a value which falls within a range of from 1600 to 3000 rpm, for example. When the ventricular assist blood pump according to the present invention is a magnetic floating type ventricular assist blood pump, it is unnecessary for the rotary part to include a seal ring, a cushion ring and a rotary shaft.

As shown in FIG. 3 and FIGS. 4A, 4B and 4C, the impeller 12 is connected to the rotary shaft 18, and is rotatable about an axis of rotation (an axis of rotation ax of the impeller 12). As shown in FIGS. 4A, 4B and 4C, with respect to the impeller 12 in the embodiment, four blades 12b protrude from a portion of the impeller 12 in the vicinity of the axis of rotation ax (impeller proximal portion 12a). The impeller proximal portion 12a is rotationally symmetrical about the axis of rotation ax of the impeller.

A suction port 34 and a discharge port 36 are formed in the housing 30. The suction port 34 is disposed on the axis of rotation ax of the impeller 12, and the discharge port 36 is disposed on an outer peripheral side of the impeller 12 (a portion of an inner wall surface of the housing 30 which opposedly faces an outer edge portion of the blades 12b of the impeller 12). In FIGS. 2A and 2B, symbol 32 indicates a body of the housing 30 (housing body).

In the ventricular assist blood pump 110, the suction port 34 is a hole formed on the axis of rotation ax of the impeller 12 of the pump chamber 31. In the ventricular assist blood pump 110, a sleeve-shaped discharge path 37 is attached to the discharge port 36. In the ventricular assist blood pump of the present invention, a sleeve-shaped suction path may be attached also to the suction port. The discharge path may not be attached to the discharge port. Further, a structure for mounting a suction path or the like may be disposed in the vicinity of the suction port, and a structure for mounting a discharge path or the like may be disposed in the vicinity of the discharge port.

In the ventricular assist blood pump 110 according to the embodiment, a numerical value obtained by dividing an outer profile volume of the rotary part 10 by a rotary volume of the rotary part 10 is set to a value which falls within a range of from 0.1 to 0.4. It is more preferable that the numerical value obtained by dividing the outer profile volume of the rotary part 10 by the rotary volume of the rotary part 10 be set to 0.2 or more. Further, it is more preferable that the numerical value obtained by dividing the outer profile volume of the rotary part 10 by the rotary volume of the rotary part 10 be set to 0.3 or less.

In this specification, “outer profile volume” of a certain constitutional element means a volume which is defined by an outer peripheral surface of a portion of the constitutional element which is exposed to a pump chamber. In a case where a space exists in a constitutional element and the space is not exposed to the pump chamber, a volume which is calculated while assuming that the space does not exist is set as the outer profile volume.

In this specification, “rotary part” means a volume of a space which is obtained by sweeping of the rotary part when the rotary part is rotated. With respect to the rotary volume of the rotary part, a volume of a part or a portion which is rotationally symmetrical with respect to the axis of rotation such as an impeller proximal portion is also included in the rotary volume of the rotary part.

In this embodiment, the outer profile volume and the rotary volume of the rotary part also include volumes of the seal ring 14 and the cushion ring 16. In this embodiment, a lower end of the seal ring 14 forms a lower end of a rotationally symmetrical portion of the rotary part 10.

The rotary shaft 18 is not exposed in the pump chamber 31 in this embodiment and hence, the rotary shaft 18 is irrelevant to the calculation of the outer profile volume and the rotary volume.

In the ventricular assist blood pump 110 according to the embodiment, assuming a plane which is perpendicular to the axis of rotation ax of the impeller and includes a distal end of the rotary part 10 on a suction port 34 side during an operation time of the ventricular assist blood pump 110 as a predetermined plane P and assuming a volume which is a portion of a volume of the pump chamber 31 disposed on a side where the rotary part 10 exists and is obtained by partitioning by the predetermined plane P as a pump chamber main volume, a numerical value obtained by dividing the rotary volume of the rotary part 10 by the pump chamber main volume is set to a value which falls within a range of from 0.4 to 0.7. It is preferable that the numerical value obtained by dividing the rotary volume of the rotary part 10 by the pump chamber main volume be set to 0.5 or more. It is more preferable that the numerical value obtained by dividing the rotary volume of the rotary part 10 by the pump chamber main volume be set to 0.65 or less.

Further, in the ventricular assist blood pump 110 according to the embodiment, the numerical value obtained by dividing the outer profile volume of the rotary part 10 by the pump chamber main volume is set to a value which falls within a range of from 0.05 to 0.2. It is more preferable that the numerical value obtained by dividing the outer profile volume of the rotary part 10 by the pump chamber main volume be set to 0.1 or more. It is still more preferable that the numerical value obtained by dividing the outer profile volume of the rotary part 10 by the pump chamber main volume be set to 0.17 or less.

The reason that the condition “during an operation time of the ventricular assist blood pump 110” is set with respect to “a distal end of the rotary part on a suction port side” which becomes a reference for the predetermined plane is that the position of the rotary part (particularly, the impeller) differs between during an operation time and a non-operation time. In this specification, “operation time” is a period in which the ventricular assist blood pump is actually used in a body of a user or during a period in which a similar operation is performed (for example, during a period in which the ventricular assist blood pump is operated on a trial basis outside the body of the user on a condition set by estimating an actual use of the ventricular assist blood pump).

In this specification, volumes of constitutional elements attached to the suction port and the discharge port (the discharge path, the suction path and the structure for mounting the discharge path and the suction path) are not included in “volume of the pump chamber” and “pump chamber main volume”.

In a case where the discharge port is formed on a curved surface of an inner wall of the housing, the volume of the pump chamber and the pump chamber main volume are calculated assuming the existence of a wall surface which corresponds to an imaginary surface obtained by smoothly connecting an inner wall of the housing to the curved surface (an inner wall surface of the housing in a state where the discharge port is smoothly closed by the inner wall surface).

In this specification, “volume of the pump chamber” and “pump chamber main volume” include a volume of a portion where the rotary part exists. However, a space which allows the rotary shaft to pass therethrough is not included in the volume of the pump chamber and the pump chamber main volume in this specification. That is, in calculating a volume of the pump chamber and the pump chamber main volume, it is assumed that there is no space which allows the rotary shaft to pass therethrough (a planar inner wall exists on an edge of the space which allows the rotary shaft passes therethrough on a housing side).

In the ventricular assist blood pump 110 according to the embodiment, a numerical value obtained by dividing an outer profile volume of the blades 12b of the impeller 12 by a rotary volume of the blades 12b is set to a value which falls within a range of from 0.05 to 0.2.

With such a configuration, the numerical value obtained by dividing the outer profile volume of the blades 12b of the impeller 12 by the rotary volume of the blades 12b is 0.05 or more. Accordingly, a rotational speed of the rotary part 10 for acquiring a required flow rate can be reduced. Further, the value is 0.2 or less and hence, the blades 12b can ensure a sufficient strength.

In this specification, “outer profile volume of the blades of the impeller” means an outer profile volume obtained by excluding a portion which is rotationally symmetrical with respect to the axis of rotation (for example, impeller proximal portion) from the outer profile volume of the whole impeller.

In this specification, “the rotary volume of the blades of the impeller” means a volume of a space which is obtained by sweeping of the blades of the impeller when the rotary part is rotated.

In the ventricular assist blood pump 110 according to the embodiment, a numerical value obtained by dividing a rotary volume of the blades 12b by a pump chamber main volume is set to a value which falls within a range of from 0.35 to 0.6.

With such a configuration, the numerical value obtained by dividing the rotary volume of the blades 12b by the pump chamber main volume is 0.35 or more and hence, a rotational speed of the rotary part 10 for acquiring a required flow rate can be reduced. Further, the numerical value is 0.6 or less and hence, it is possible to give a margin to volume of a portion of the inside of the pump chamber 31 through which the blades 12b do not pass and hence, it is possible to sufficiently reduce a stress applied to blood sucked into the ventricular assist blood pump 110.

In the ventricular assist blood pump 110 according to the embodiment, a numerical value obtained by dividing the outer profile volume of the blades 12b by the pump chamber main volume is set to a value which falls within a range of from 0.02 to 0.1.

With such a configuration, the numerical value obtained by dividing the outer profile volume of the blades 12b by the pump chamber main volume is set to 0.02 or more. Accordingly, a pressure loss can be sufficiently reduced and hence, it is possible to sufficiently increase a change in a flow rate against a change in a head. Further, the value is 0.1 or less and hence, it is possible to ensure a sufficient flow rate (allowing the blades 12b to have a sufficient force to move blood).

In the ventricular assist blood pump 110 according to the embodiment, a numerical value obtained by dividing the outer profile volume of the blades 12b by an outer profile volume of the rotary part 10 is set to a value which falls within a range of from 0.3 to 0.5.

With such a configuration, the numerical value obtained by dividing the outer profile volume of the blades 12b by the outer profile volume of the rotary part 10 is 0.3 or more. Accordingly, a volume of a portion of the rotary part 10 excluding the blades 12b can be sufficiently reduced and hence, blood discharging efficiency of the ventricular assist blood pump 110 can be increased. Further, the numeral value is 0.5 or less and hence, it is possible to suppress the occurrence of a case where an excessively large load is applied to the blades 12b so that the blades 12b are damaged.

In the ventricular assist blood pump 110 according to the embodiment, a numerical value obtained by dividing an outer profile volume of the blades 12b by a rotary volume of the rotary part 10 is set to a value which falls within a range of from 0.05 to 0.2.

With such a configuration, the numerical value obtained by dividing an outer profile volume of the blades 12b by the rotary volume of the rotary part 10 is 0.2 or less and hence, a rotational speed of the rotary part 10 for acquiring a required flow rate can be reduced. Further, the numerical value is 0.05 or more and hence, the blades 12b can ensure a sufficient strength.

In the ventricular assist blood pump 110 according to the embodiment, a numerical value obtained by dividing the rotary volume of the blades 12b by the rotary volume of the rotary part 10 is set to a value which falls within a range of from 0.8 to 0.9.

With such a configuration, the numerical value obtained by dividing the rotary volume of the blades 12b by the rotary volume of the rotary part 10 is 0.8 or more. Accordingly, a volume of a portion of the rotary part 10 excluding the blades 12b can be sufficiently reduced and hence, blood discharging efficiency of the ventricular assist blood pump 110 can be enhanced. Further, the numerical value is 0.9 or less and hence, it is possible to prevent the occurrence of a case where an excessively large load is applied to a joining portion between the blades 12b and other portions or the like.

In the ventricular assist blood pump 110 according to the embodiment, a minimum distance between the impeller 12 and an inner wall of the housing 30 during the operation of the ventricular assist blood pump is set to a value which falls within a range of from 0.2 mm to 1.0 mm.

In the ventricular assist blood pump 110 according to the embodiment, a minimum distance between the impeller 12 and the base part 20 during the operation of the ventricular assist blood pump 110 is set to a value which falls within a range of from 0.1 mm to 0.9 mm.

In the ventricular assist blood pump 110 according to the embodiment, in a measurement of a pressure loss where a liquid having viscosity and density equivalent to viscosity and density of blood is used as a working liquid and a flow rate is set to 6 L/min in a state where the ventricular assist blood pump 110 is stopped, the pressure loss is 20 mmHg or less.

Hereinafter, advantageous effects acquired by the ventricular assist blood pump 110 according to the embodiment are described hereinafter.

According to the ventricular assist blood pump 110 of this embodiment, the numerical value obtained by dividing the outer profile volume of the rotary part 10 by the rotary volume of the rotary part 10 is 0.4 or less and hence, an efficiency that the ventricular assist blood pump 110 discharges blood can be increased by making the outer profile volume of the rotary part 10 sufficiently small whereby a rotational speed of the rotary part 10 for obtaining a required flow rate can be reduced. As a result, the ventricular assist blood pump 110 of this embodiment becomes a ventricular assist blood pump which can suppress a degree of worsening of a health condition of a user when the ventricular assist blood pump is used for a long period compared to a conventional ventricular assist blood pump.

According to the ventricular assist blood pump 110 of this embodiment, a numerical value obtained by dividing the outer profile volume of the rotary part 10 by the rotary volume of the rotary part 10 is 0.1 or more and hence, the impeller 12 can sufficiently ensure a strength.

According to the ventricular assist blood pump 110 of this embodiment, a numerical value obtained by dividing the rotary volume of the rotary part 10 by the pump chamber main volume is 0.4 or more. Accordingly, the rotary volume of the rotary part 10 with respect to the pump chamber main volume can be sufficiently increased and hence, blood discharging efficiency of the ventricular assist blood pump 110 can be increased whereby a rotational speed of the rotary part 10 for acquiring a required flow rate can be reduced. As a result, the ventricular assist blood pump of this embodiment becomes a ventricular assist blood pump which can suppress a degree of worsening of a health condition of a user when the ventricular assist blood pump is used for a long period compared to a conventional ventricular assist blood pump.

Further, according to the ventricular assist blood pump 110 of this embodiment, a numerical value obtained by dividing the rotary volume of the rotary part 10 by the pump chamber main volume is 0.7 or less. Accordingly, a ratio that the rotary part 10 occupies a main part of the pump chamber 31 during an in-use time of the ventricular assist blood pump 110 can be set to a proper value and hence, in the inside of the pump chamber 31, a volume of a portion which the rotary part 10 does not occupy and a volume of a portion through which the rotary part 10 does not pass can have a margin respectively. Accordingly, a stress applied to sucked blood can be sufficiently reduced, and it is possible to prevent the flow of blood from the suction port 34 to the discharge port 36 from being obstructed by the rotary part 10 itself. As a result, the ventricular assist blood pump 110 can be operated in a stable manner.

According to the ventricular assist blood pump 110 of this embodiment, the numerical value obtained by dividing the outer profile volume of the rotary part 10 by the pump chamber main volume is set to 0.2 or less and hence, a pressure loss can be sufficiently reduced whereby it is possible to sufficiently increase a change in a flow rate against a change in a head. As a result, the ventricular assist blood pump 110 of this embodiment becomes a ventricular assist blood pump which can suppress a degree of worsening of a health condition of a user when the ventricular assist blood pump 110 is used for a long period compared to a conventional ventricular assist blood pump.

Further, in the ventricular assist blood pump 110 according to this embodiment, the numerical value obtained by dividing the outer profile volume of the rotary part 10 by the pump chamber main volume is set to 0.05 or more and hence, it is possible to sufficiently ensure a flow rate of blood (to sufficiently ensure a force which the rotary part 10 moves blood).

According to the ventricular assist blood pump 110 of this embodiment, the minimum distance between the impeller 12 and the housing 30 during the operation of the ventricular assist blood pump 110 is 0.2 mm or more and hence, a pressure loss can be sufficiently lowered. Further, since the minimum distance is 1.0 mm or less, it is possible to more sufficiently ensure a force generated by the impeller 12 for discharging blood.

According to the ventricular assist blood pump 110 of this embodiment, the minimum distance between the impeller 12 and the housing 30 during the operation of the ventricular assist blood pump 110 is 0.2 mm or more and hence, it is possible to suppress the occurrence of a case where a foreign substance (for example, thrombus) is nipped between the impeller 12 and the housing 30 so that the rotation of the impeller 12 is impeded. As a result, the operational stability of the ventricular assist blood pump 110 can be further improved.

In the ventricular assist blood pump 110 according to the embodiment, the minimum distance between the impeller 12 and the base part 20 during the operation of the ventricular assist blood pump 110 is 0.1 mm or more and hence, a pressure loss can be sufficiently reduced, and the minimum distance is 0.9 mm or less and hence, it is possible to sufficiently ensure a force generated by the impeller 12 for discharging blood.

In the ventricular assist blood pump 110 according to the embodiment, in a measurement of a pressure loss where a liquid having viscosity and density equivalent to viscosity and density of blood is used as a working liquid and a flow rate is set to 6 L/min in a state where the ventricular assist blood pump 110 is stopped, the pressure loss is 20 mmHg or less. Accordingly, a pressure loss can be sufficiently reduced.

In the ventricular assist blood pump 110 according to the embodiment, the ventricular assist blood pump 110 is a centrifugal ventricular assist blood pump and hence, the ventricular assist blood pump 110 can more efficiently suck and discharge blood compared to an axial-flow ventricular assist blood pump.

Although the present invention has been described with reference to the above-mentioned embodiment, the present invention is not limited the above-mentioned embodiment. Various modifications are conceivable in various modes without departing from the gist of the present invention. For example, the following modifications are conceivable.

(1) Sizes, the numbers, materials, shapes and the like of the respective constitutional elements described in the above-mentioned embodiment and depicted in the respective drawings are provided only for an exemplifying purpose, and can be changed within a scope that advantageous effects of the present invention are not jeopardized.
(2) The above-mentioned ventricular assist blood pump 110 according to the embodiment includes three technical features, that is, “a numerical value obtained by dividing the outer profile volume of the rotary part 10 by the rotary volume of the rotary part 10 is set to a value which falls within a range of from 0.1 to 0.4”, “a numerical value obtained by dividing the rotary volume of the rotary part 10 by the pump chamber main volume is set to a value which falls within a range of from 0.4 to 0.7”, and “a numerical value obtained by dividing the outer profile volume of the rotary part 10 by the pump chamber main volume is set to a value which falls within a range of from 0.05 to 0.2”. However, the present invention is not limited to such a configuration. A ventricular assist blood pump which includes one or two technical features out of the above-mentioned three technical features is also included in the scope of the present invention.

1. A ventricular assist blood pump comprising:
a rotary part which has an impeller;
a base part which rotatably supports the rotary part; and
a housing which forms a pump chamber which houses the rotary part in combination with the base part, wherein
a numerical value obtained by dividing an outer profile volume of the rotary part by a rotary volume of the rotary part is set to a value which falls within a range of from 0.1 to 0.4.

2. A ventricular assist blood pump comprising:
a rotary part which has an impeller;
a base part which rotatably supports the rotary part; and
a housing which forms a pump chamber which houses the rotary part in combination with the base part, wherein
a suction port which is disposed on an axis of rotation of the impeller and a discharge port which is disposed on an outer peripheral side of the impeller are formed in the housing, and
assuming a plane which is perpendicular to the axis of rotation of the impeller and includes a distal end of the rotary part on a suction port side during an operation time of the ventricular assist blood pump as a predetermined plane and assuming a volume which is a portion of a volume of the pump chamber disposed on a side where the rotary part exists and is obtained by partitioning by the predetermined plane as a pump chamber main volume,
a numerical value obtained by dividing a rotary volume of the rotary part by the pump chamber main volume is set to a value which falls within a range of from 0.4 to 0.7.

3. A ventricular assist blood pump comprising:
a rotary part which has an impeller;
a base part which rotatably supports the rotary part; and
a housing which forms a pump chamber which houses the rotary part in combination with the base part, wherein
a suction port which is disposed on an axis of rotation of the impeller and a discharge port which is disposed on an outer peripheral side of the impeller are formed in the housing, and
assuming a plane which is perpendicular to the axis of rotation of the impeller and includes a distal end of the rotary part on a suction port side during an operation time of the ventricular assist blood pump as a predetermined plane and assuming a volume which is a portion of a volume of the pump chamber and is disposed on a side where the rotary part exists obtained by partitioning by the predetermined plane as a pump chamber main volume,
a numerical value obtained by dividing an outer profile volume of the rotary part by the pump chamber main volume is set to a value which falls within a range of from 0.05 to 0.2.

4. The ventricular assist blood pump according to claim 1, wherein a minimum distance between the impeller and the housing during the operation of the ventricular assist blood pump is set to a value which falls within a range of from 0.2 mm to 1.0 mm.

5. The ventricular assist blood pump according to claim 1, wherein a minimum distance between the impeller and the base part during the operation of the ventricular assist blood pump is set to a value which falls within a range of from 0.1 mm to 0.9 mm.

6. The ventricular assist blood pump according to claim 1, wherein in a measurement of a pressure loss where a liquid having viscosity and density equivalent to viscosity and density of blood is used as a working liquid and a flow rate is set to 6 L/min in a state where the ventricular assist blood pump is stopped, the pressure loss is 20 mmHg or less.

7. The ventricular assist blood pump according to claim 1, wherein the ventricular assist blood pump is a centrifugal ventricular assist blood pump.

8. The ventricular assist blood pump according to claim 2, wherein a minimum distance between the impeller and the housing during the operation of the ventricular assist blood pump is set to a value which falls within a range of from 0.2 mm to 1.0 mm.

9. The ventricular assist blood pump according to claim 2, wherein a minimum distance between the impeller and the base part during the operation of the ventricular assist blood pump is set to a value which falls within a range of from 0.1 mm to 0.9 mm.

10. The ventricular assist blood pump according to claim 2, wherein in a measurement of a pressure loss where a liquid having viscosity and density equivalent to viscosity and density of blood is used as a working liquid and a flow rate is set to 6 L/min in a state where the ventricular assist blood pump is stopped, the pressure loss is 20 mmHg or less.

11. The ventricular assist blood pump according to claim 2, wherein the ventricular assist blood pump is a centrifugal ventricular assist blood pump.

12. The ventricular assist blood pump according to claim 3, wherein a minimum distance between the impeller and the housing during the operation of the ventricular assist blood pump is set to a value which falls within a range of from 0.2 mm to 1.0 mm.

13. The ventricular assist blood pump according to claim 3, wherein a minimum distance between the impeller and the base part during the operation of the ventricular assist blood pump is set to a value which falls within a range of from 0.1 mm to 0.9 mm.

14. The ventricular assist blood pump according to claim 3, wherein in a measurement of a pressure loss where a liquid having viscosity and density equivalent to viscosity and density of blood is used as a working liquid and a flow rate is set to 6 L/min in a state where the ventricular assist blood pump is stopped, the pressure loss is 20 mmHg or less.

15. The ventricular assist blood pump according to claim 3, wherein the ventricular assist blood pump is a centrifugal ventricular assist blood pump.
